(19) United States
(12) Patent Application Publication
(10) Pub. No.: US 2006/0282031 A1
(43) Pub. Date: Dec. 14, 2006

(54) Scented orthopedic device

(76) Inventor: Michael A. Gleiber, Edgewater, NJ (US)

Correspondence Address: AKERMAN SENTERFITT, P.O. BOX 3188, WEST PALM BEACH, FL, 33402-3188, US

(21) Appl. No.: 11/338,297

(22) Filed: Jan. 24, 2006

Related U.S. Application Data
(60) Provisional application No. 60/689,831, filed on Jun. 13, 2005.

(51) Int. Cl. A61F 5/00

(52) U.S. Cl. 602/14

(57) ABSTRACT

A scented orthopedic device that substantially reduces or eliminates the odors associated with wearing of the orthopedic device, such as those associated with casts that are worn for extended periods of time and/or produce malodors during use. These scented orthopedic devices include a scented article that is added to the orthopedic device in a manner capable of permitting scents from the scented article to mask and/or eliminate any odors associated with the orthopedic device. The scented article may be applied in a variety of different manners and locations, may be combined with the use of a compatible coloring agent, and/or may use one or more scents and/or scent strengths.

### CROSS-REFERENCE TO RELATED APPLICATIONS

This application claims the benefit of U.S. Provisional Application No. 60/689,831, filed in the United States Patent and Trademark Office on Jun. 13, 2005, the entirety of which is incorporated herein by reference.

### FIELD OF THE INVENTION

The present invention is directed to orthopedic devices. In particular, the present invention is directed to scented orthopedic devices, such as casts.

### BACKGROUND OF THE INVENTION

Many people receive injuries such that they need an orthopedic device, namely, a cast. Serious injuries require that the person wear the device for an extended period of time. In many instances, such as those injuries involving broken bones and/or dislocated joints, the person may be required to wear a cast for several months, or even longer.

The function of a cast is to rigidly protect an injured bone or joint. It serves to hold a broken bone in proper alignment to prevent it from moving while it heals. Casts may also be used to help rest a bone or joint to relieve pain that is caused by moving it (such as when a severe sprain occurs, but no broken bones). As a result, however, casts must be rigid and worn for extended periods of time. In addition, due to possible infection of the skin should the skin under a cast become wet, wearers of casts are specifically instructed to keep the cast dry. Unfortunately, this prevents the wearer from cleaning the skin under the cast.

Since a wearer is unable to clean the skin under the cast, bacteria under the cast can grow unchecked, leading to the formation of malodors that can make the cast unpleasant to wear. This may result in a situation wherein the wearer of the cast is subjected to malodors for extended periods of time such that the wearer may attempt to clean the area, contrary to the instructions provided, which may lead to more dangerous situations, such as infections, should the skin become wet. Alternatively, the wearer of the cast may be forced to remain secluded should the malodors be offensive to those nearby, such as individuals in a public place.

Should a person ignore requests and attempt to clean under a cast, the person risks damaging the skin and/or causing an infection, which would necessitate removal of the cast for cleaning of the area before reapplying the cast. However, removal of the cast prior to the complete healing of the bone or joint increases the risk that the bone might shift, which would require surgery to fix the injury.

Accordingly, a scented orthopedic device can help to reduce or eliminate odors associated with orthopedic devices, such as casts. While at least one company markets a scented cast, such product is more a marketing gimmick than a useful product that actually reduces odors over the lifetime of the cast. The product on the market does not utilize any effective time/extended release mechanism that will gradually display the scent over a period of time to combat odors that will otherwise develop.

Further, it is important to develop a product that will increase cast care compliance to help reduce the risk of infections. The scented cast now on the market has minimal value in this respect. However, if scent and color are combined in a cast, a product is developed that creates greater interest, particularly with pediatric patients, to create a sense of ownership which in turn, enhances medical compliance.

### SUMMARY OF THE INVENTION

As such, the present invention provides a scented orthopedic device that substantially reduces and/or eliminates malodors associated with wearing of an orthopedic device, such as those malodors associated with casts that are worn for extended periods of time. These scented orthopedic devices include a scented article that is integrated with the orthopedic device in a manner capable of permitting scents from the scented article to mask and/or eliminate any odors associated with the orthopedic device. The scented article may be applied in a variety of different manners and locations, and/or may use one or more scents and/or scent strengths. In particular, the scented article will incorporate a time/extended release mechanism to gradually display the scent.

In addition, in those embodiments wherein the orthopedic device is a cast, the systems of the present invention help patients to comply with proper cast care. In these embodiments, the malodors associated with extended wearing of the cast are masked, reduced and/or eliminated through the use of a time/extended release mechanism to gradually display the scent such that the patient is less inclined to attempt to clean under the cast, thereby reducing the risk of infection and/or surgery, which has an added benefit of reducing costs associated with injuries resulting in a cast being used.

In particular, the present invention provides a scented orthopedic device including an orthopedic device and a scented article applied to the orthopedic device. In another embodiment, the orthopedic device is a cast and the scented article is selected from a spray, a scented adhesive compound, a scented strip, a scented sticker, a scented polymer film, or a combination thereof. The scent may be selected from a variety of different pleasant fragrances, such as fruit scents, floral scents, herbal scents, and other natural and synthetic scents. The scent is released through an extended release mechanism that gradually displays the scent over time.

In a further embodiment, the scent is matched with a particular color of cast. For example, green casts may smell like mint, yellow casts like lemon, purple casts like lilac, etc. Such uniqueness will create a stronger feeling of ownership particularly in certain segments of the population such as the pediatric group. The greater ownership will enhance medical compliance, which in turn will serve to reduce infectious and unsanitary conditions. These and other embodiments are described in greater detail below.

Reduction of the malodors may also be facilitated by a combination approach of adding a scent to both the skin to be covered by the cast and to the cast itself.

### BRIEF DESCRIPTION OF THE DRAWINGS

Other objects, features and advantages of the present invention will become apparent upon reading the following detailed description, while referring to the attached drawings, in which:

FIG. 1 is a depiction of a scented cast according to one embodiment of the present invention.

### DETAILED DESCRIPTION OF THE INVENTION

The present invention is more particularly described in the following examples that are intended to be illustrative only since numerous modifications and variations therein will be apparent to those skilled in the art. As used in the specification and in the claims, the singular form “a,” “an,” and “the” may include plural referents unless the context clearly dictates otherwise. Also, as used in the specification and in the claims, the term “comprising” may include the embodiments “consisting of” and “consisting essentially of.”

The present invention provides a scented orthopedic device capable of substantially reducing and/or eliminating malodors associated with orthopedic devices, such as those associated with casts that are worn for extended periods of time and/or produce malodors during use. These scented orthopedic devices include a scented article that is added to the orthopedic device in a manner capable of permitting scents from the scented article to mask and/or eliminate any odors associated with the orthopedic device. The scented article may be applied under the orthopedic device, on an external surface of the orthopedic device, and/or in an interior portion of the orthopedic device. The scented article may be added to the orthopedic device prior to application of the orthopedic device to the wearer, during application of the orthopedic device to the wearer, or after the orthopedic device has been put on the wearer.

Accordingly, in one aspect of the present invention, the scented orthopedic devices include the use of a scented article. The scented article may be any article capable of holding and/or releasing a scent that may be detected by a wearer of an orthopedic device and/or any persons nearby, thereby reducing and/or eliminating the malodors associated with wearing of the orthopedic device, such as a cast.

The ability to impregnate scents into a scented article may use a wide variety of technologies. In one embodiment, scented and/or aromatic oils may be embedded into capsules and may be integrated into a sticker, polymer film, spray or printable ink that releases scent. Some of the more traditional technologies require touch for the smell to be activated, whereas new technologies that use slightly different encapsulation technologies may be used to release an ongoing smell.

In one embodiment of the present invention, the scented article is a polymer film having a scented material embedded therein. In one embodiment, the scented material is an encapsulated oil particle that is embedded within the polymer film. In this embodiment, the scent may be provided in a scented oil or other scented liquid which is then encapsulated and then embedded into a polymer film. The encapsulated material may be embedded during or after formation of the polymer film. In addition, any other technology capable of incorporating a scented material into a polymer film may also be used in the present invention. Any polymer film capable of being wrapped around an arm, a cast or other orthopedic device or otherwise integrated with a cast may be used in the present invention. Examples of polymer films include, but are not limited to, polyethylene, polypropylene, polyethylene glycol (PEG), polyethylene terephalate (PET), polyvinyl alcohol (PVOH), or a combination thereof.

In another embodiment of the present invention, the scented article is a woven or nonwoven fabric material having a scented material integrated therewith. In one embodiment, the scented material is located in the spaces between the fibers composing the woven or nonwoven fabric. In another embodiment, the scented material is embedded within the fibers composing the woven or nonwoven fabric. The fabric may comprise natural or synthetic fibers. The fabric may be any fabric capable of being wrapped around an arm, a cast or other orthopedic device or otherwise integrated with a cast.

In yet another embodiment of the present invention, the scented article is a scented sticker that may have an adhesive material on one side of the scented sticker for attachment to the orthopedic device. The scented sticker would also include a scented material that is integrated with the sticker substrate. One example for a method of forming a scented substrate is using the technology available from Disperse Technologies. In this embodiment, a controlled-release technology is used to trap any kind of oil-soluble scent into a powder coating. The powdered coating is encapsulated and applied is a thin film, which is then UV-cured technology, to apply a smell to any number of board and paper applications, such a sticker substrates that are then capable of being attached to an orthopedic device.

In one embodiment, the sticker may release the smell automatically. In another embodiment, the present invention may utilize a “scratch-and-sniff” type of scented article wherein the wearer of the cast, when the malodors become stronger, may activate the scented article by scratching the sticker to release the scent to reduce the malodors. One example of such a scented strip is one made using a MicroFragrance® Scratch ‘n’ Sniff sticker available from Arcade Marketing (New York, N.Y.). Other examples include the DiscCover® label, a scented, adhesive label and AromaLacquer®, is a varnish system that, when rubbed, delivers a selected scent. Both are also available from Arcade Marketing.

In still another embodiment, the scented article is a scented spray that includes a liquid scent material that will substantially adhere to the orthopedic device after spraying onto the device. The liquid scent may comprise, in one embodiment, a scented particle embedded in a liquid carrier. In another embodiment, the liquid scent may comprise a scented liquid that may be applied alone or in addition to a liquid carrier. In one embodiment, the scented spray may take the form of a printable ink. One example of a scented printable ink is by Scentisphere® called Rub'NSmell®, a printable, scented ink that may be applied directly to the cast or other orthopedic device.

In yet another embodiment, the scented article may comprise a scented adhesive material. In these embodiments, the scented adhesive may be used alone or in conjunction with another article, such as a sticker, fabric or polymer film, that also includes a scent.

In still another embodiment, the scented article may comprise a cast material having the scented material imbedded therein. As such, the plaster or fiberglass or any other cast material would include the scented material embedded therein in a manner similar to those previously described in regards to the scented polymer film or in any other manner capable of integrating a scent with a cast material.

The scent may be selected from any scent or flavor such as floral, fruit, or herbal scents including, but not limited to, cherry, apple, lime, orange, jasmine, rose, lavender, petitgrain, patchouli, juniper, ginger, orange blossom, tangerine, rosemary, peppermint, wintergreen, spearmint, peach blossom, eucalyptus, lemon, balsam fir, bergamot, ylang, neroli, sandalwood, chamomile, geranium, musk, frankincense, cedar, plum, mandarin, pine, tea tree, clary sage, vetiver, cypress, coriander, grapefruit, grape, bergamot, cordamom, basil, lilac, everlasting, lily, lily of the valley, or a combination thereof. In each case, the scent may be matched with an associated color to create greater excitement in the wearer to improve the acceptability of the cast. It must be recognized that many, particularly in the pediatric group, are going to have some trepidation to having a foreign hard material wrapped around their body. If a child arrives at school with a pink cast that smells like bubblegum, her injury may be more “interesting and accepted” compared with a colorless cast smelling of mold and mildew.

Controlled delivery systems such as disclosed in U.S. Pat. No. 6,531,444 may have particular utility in applying fragrance to cast materials. Also, the delivery system of Published U.S. Patent Application No. 2003/0203829, which discloses a delivery system which can utilize free-flowing powder formed of solid hydrophobic, nano-spheres of ingredients such as fragrance, is another system which can be useful in applying fragrance to casts.

Systems such as those disclosed in U.S. Patent Application No. 2003/0198680 which provide high-impact fragrance in response to moisture, are useful in the step of applying a cast to the body.

The targeted controlled delivery system of U.S. Patent Application No. 2003/0195133, which relates to applying fragrances to the skin, can be used in conjunction with the application of fragrances to the cast to apply fragrances to the skin beneath the cast.

The present invention also provides an orthopedic device kit that includes a plurality of different scented articles. For example, in those instances wherein the orthopedic device, such as a cast, may be worn for extended periods of time, the malodors associated with wearing of the cast will increase in strength as time progresses. As such, in one embodiment, it may be beneficial for the kit to include replacement scented article to replace a scented article that has begun to lose its scent over time.

Alternatively, or in addition thereto, the kit may include a plurality of scented articles having different strengths of scent associated therewith. In these embodiments, as the strength of the malodors increases, stronger smelling scented articles may be applied. Alternatively, in lieu of stronger scents, the kit may instruct the wearer to wear additional scented articles as time progresses. For example, if the scented article were a scented strip, the kit may include instructions such that, for the first week, one scented strip is to be applied. For the second week, two scented strips are to be applied and so on. It is to be understood, however, that the number of strips to be applied and the time for applying these strips may vary and the present invention is not limited to a strip per week basis.

In another embodiment, the kit may provide a plurality of scented articles each having the same or a different scent associated therewith. In these embodiments, the individual may decide to change the scent of their orthopedic device at some point in the future and the kit would provide alternative scented articles for permitting the individual to change the scent whenever they desired.

The concepts of the present invention may be used in conjunction with any orthopedic device, such as prosthetic devices, prosthetic liners, and casts. In one embodiment of the present invention, the orthopedic device is a cast. The present invention may be used with any type of cast material including, but not limited to, plaster casts and fiberglass casts.

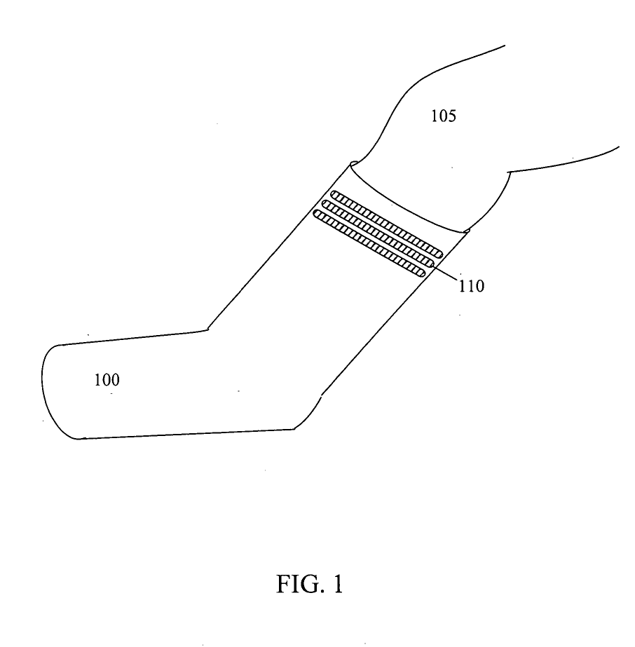

Reference is now made to FIG. 1, which provides one embodiment of the present invention. It is to be understood, however, that FIG. 1 is only one embodiment of the present invention. In this embodiment, a cast 100 is placed on a patient 105, such as on the patient's leg. The scented article in this embodiment is a scented strip 110 that includes a scented material therein. In this embodiment, three scented strips 110 are provided, although it is to be understood that more or fewer than three strips may be used.

Although the illustrative embodiments of the present disclosure have been described herein with reference to the accompanying drawings and examples, it is to be understood that the disclosure is not limited to those precise embodiments, and various other changes and modifications may be affected therein by one skilled in the art without departing from the scope or spirit of the disclosure. All such changes and modifications are intended to be included within the scope of the disclosure as defined by the appended claims.

What is claimed is:

1. A scented orthopedic device comprising:
an orthopedic device; and
a long lasting scent integrated with the orthopedic device.

2. The scented orthopedic device of claim 1, wherein the orthopedic device comprises a cast.

3. The scented orthopedic cast of claim 2, including a time release mechanism for the scent.

4. The scented orthopedic cast of claim 3, wherein the scented article is selected from a scented spray, a scented adhesive compound, a scented strip, a scented sticker, a scented polymer film, a scented fabric, or a combination thereof.

5. The scented orthopedic cast of claim 3, wherein the scented article includes a scent selected from cherry, apple, lime, orange, jasmine, rose, lavender, petitgrain, patchouli, juniper, ginger, orange blossom, tangerine, rosemary, peppermint, wintergreen, spearmint, peach blossom, eucalyptus, lemon, balsam fir, bergamot, ylang, neroli, sandalwood, chamomile, geranium, musk, frankincense, cedar, plum, mandarin, pine, tea tree, clary sage, vetiver, cypress, coriander, lemon, grapefruit, bergamot, cordamom, basil, lilac, everlasting, lily, lily of the valley, or a combination thereof.

6. The scented orthopedic cast of claim 6, wherein the cast is of a compatible color with the scent.

7. The scented orthopedic device of claim 1, wherein the orthopedic device is a cast and the scented article comprises a scented material integrated with a material used to form the cast.

8. The method of improving the appeal of wearing a cast, comprising the steps of:
applying a long-lasting scent to the cast;
applying a color that is compatible to the scent to the cast; and
applying a scent to the skin beneath the cast.

9. The method of minimizing malodors associated with wearing a cast, comprising the steps of:
applying a scent to the skin where the cast is to be placed prior to putting the cast in position; and
applying a long-lasting compatible scent to the cast as it is applied to the body.
